(19) United States
(12) Patent Application Publication
(10) Pub. No.: US 2008/0194662 A1
(43) Pub. Date: Aug. 14, 2008

(54) CLEANSER COMPOSITION

(76) Inventor: Audrey Kunin, Mission Hills, KS (US)

Correspondence Address: HUSCH BLACKWELL SANDERS LLP, 4801 Main Street, Suite 1000, KANSAS CITY, MO, 64112, US

(21) Appl. No.: 11/674,944

(22) Filed: Feb. 14, 2007

(51) Int. Cl. A61K 31/4164; A61K 31/315

(52) U.S. Cl. 514/385; 510/123; 514/494

(57) ABSTRACT

A cleanser composition composed of purified water, sodium laureth sulfate, zinc pyrithione, cocamidopropyl betaine, glycerol cocoate, polyquaternium-7, and a cross-linked polyacrylic acid polymer for topical application to an area affected by a skin disorder such as seborrheic dermatitis, perioral dermatitis, rosacea, acne, tinea versicolor, eczema and psoriasis.

### BACKGROUND OF THE INVENTION

Human skin is an ever-changing organ that contains many specialized cells and structures. Skin functions as a protective barrier and aids in maintaining the proper temperature for proper body functions. Skin also collects sensory information from the environment and plays an active immune system role in protecting from disease. Human skin is an amalgamation of the epidermis and the dermis. The top layer of the epidermis is the stratum corneum. This layer is the stiffest layer of the skin and is generally the layer most affected by the surrounding environment. Below the stratum corneum, is the internal layer of the epidermis. Below the epidermis, the top layer of the dermis is the papillary dermis which is composed of loose connective tissues that define the micro-relief of the skin. The reticular dermis, which is disposed beneath the papillary dermis, is a tight and connective tissue that is spatially organized. The reticular dermis is also associated with coarse wrinkles. At the bottom of the dermis lies the subcutaneous layer.

The principal functions of the skin include protection, excretion, secretion, absorption, thermoregulation, pigmentogenesis, accumulation, sensory perception, and regulation of immunological processes. These functions are detrimentally affected by, for example, dryness, yeast, and structural changes in the skin, such as due to aging and excessive sun exposure. Skin disease is a very broad term that describes numerous conditions. Some skin diseases are serious and deadly, while others are just annoying. Some skin diseases are disfiguring, while others are barely visible.

Acne is the most common skin disease treated by physicians. It is a chronic condition that affects over 85% of adolescents and young adults. There are different types of acne that respond to different types of treatment. Seborrheic dermatitis is a chronic inflammatory disorder affecting areas of the head and trunk where sebaceous glands are most prominent. Lipophilic yeasts of the Malassezia genus, as well as genetic, environmental and general health factors, contribute to this disorder. Scalp seborrhea varies from mild dandruff to dense, diffuse, adherent scale. Facial and trunk seborrhea is characterized by powdery or greasy scale in skin folds and along hair margins. Psoriasis is a lifelong skin condition caused by changes in the immune system. The rash of psoriasis is very distinctive. Rosacea is a common skin condition characterized by redness of the face and acne. Tinea versicolor is a common, benign, superficial cutaneous fungal infection usually characterized by hypopigmented or hyperpigmented macules and patches on the chest and the back. In patients with a predisposition, the condition may chronically recur. The fungal infection is localized to the stratum corneum. Perioral dermatitis is a common skin problem that mostly affects young women. Occasionally men or children are affected. Perioral refers to the area around the mouth, and dermatitis indicates redness of the skin. In addition to redness, there are usually small red bumps or pus bumps, and mild peeling. Sometimes the bumps are the most obvious feature, and the disease can look a lot like acne. The areas most affected are within the borders of the lines from the nose, to the sides of the lips, and the chin. There is frequent sparing of a small band of skin that borders the lips. Occasionally, the areas around the nose, eyes, and cheeks can be affected. Sometimes there is mild itching or burning.

All of the skin diseases discussed above predisposes the epidermis to be dry, flaky, red and inflamed. Various pharmaceuticals, including skin cleansing compositions, have been used for the treatment or prevention of these skin conditions. Patients who suffer from one or more these skin disorders are routinely prescribed prescription or over-the-counter cleansers containing one or more of the following compounds: 2% pyrithione zinc, up to 2% salicylic acid, up to 2% ketoconazole, up to 20% glycolic acid, steroids, sulfa, and coal tar distillates. These active ingredients and delivery vehicles are typically formulated to target the physiological factors responsible for creating the particular skin disorder being treated. However, these compositions further dry and inflame the skin particularly with routine and/or preventative use.

There exists therefore a need in the art for a skin cleansing composition that effectively addresses the physiology of skin diseases such as acne, seborrheic dermatitis, perioral dermatitis, rosacea, tinea versicolor, eczema and psoriasis, but does not cause a worsening of the irritation, inflammation, dryness or redness of the skin condition being treated.

### SUMMARY OF THE INVENTION

In one of many illustrative aspects of the present invention, there is provided an improved skin cleansing composition, the improved composition including purified water, sodium laureth sulfate, zinc pyrithione, cocamidopropyl betaine, glycerol cocoate, polyquaternium-7, and cross-linked polyacrylic acid polymer.

In another illustrative aspect of the present invention there is provided a method of making the improved composition of the present invention.

In yet another illustrative aspect of the present invention there is provided a method of treating skin disorders, the method comprising topically administering to an affected area a non-toxic effective dosage of the improved composition.

### DETAILED DESCRIPTION OF THE INVENTION

There is provided a novel combination of purified water, sodium laureth sulfate, zinc pyrithione, cocamidopropyl betaine, glycerol cocoate, polyquaternium-7, and a cross-linked polyacrylic acid polymer. The resulting improved composition provides a significant and unexpected reduction of irritation, inflammation, dryness and/or redness, all symptoms of certain dermatological disorders, when applied topically to an affected area. The improved composition was evaluated by clinical grading of symptoms via a nonparametric Wilcoxon Signed Rank Test. These evaluations demonstrated the mildness of the improved composition and the lack of adverse effects such as irritation, itching or stinging.

Sodium laureth sulfate, sodium lauryl ether sulfate, is a detergent and surfactant found in many personal care products (soaps, shampoos, toothpaste, etc.). It is useful in personal care products due to its excellent foaming capabilities that allows for better distribution of the product. However, known products containing sodium laureth sulfate can affect those prone to eczema and other irritants. Moreover, when rinsed off, sodium laureth sulfate, as used in known products, will have cleaned the area but will have taken moisture from the top layers of skin. In people with sensitive skin (prone to dermatitis, acne, eczema, psoriasis and chemical sensitivity) the drying property of this compound may cause flare-ups of skin conditions or may worsen existing skin conditions.

Zinc pyrithione is an antifungal agent best known for its use in treating dandruff and seborrheic dermatitis. It also has antibacterial properties and is effective against many pathogens from the streptococcus and staphylococcus class. Its antifungal effect is thought to lie in the ability of an un-ionized pyrithione molecule to disrupt membrane transport by blocking the proton pump that energizes the transport mechanism.

Cocamidopropyl betaine is a zwitterionic surfactant with a quaternary ammonium cation in its molecule. It is a viscous pale yellow transparent liquid and is often used as a surfactant in bath products like shampoos and hand soaps, and in cosmetics as an emulsifying agent and thickener, and to reduce irritation that purely ionic surfactants may cause. Cocamidopropyl betaine is a derivate of cocamide and betaine and is also known for having antiseptic properties.

Glycerol cocoate is a nonionic surfactant derived from coconut that is often used as an emulsifier and mildness additive. As a stand-alone surfactant, it has a very low foam but exceptional mildness. Glycerol cocoate also functions as a water-soluble emollient.

Polyquaternium is the designation for several polycationic polymers that are used in the personal care industry. Polyquaternium is a neologism used to emphasize the presence of quaternary ammonium centers in the polymer. Different polymers are distinguished by the numerical value that follows the word “polyquaternium.” Polyquaternium-7 is a copolymer of acrylamide and diallyldimethylammonium chloride. Polyquaterniums are positively charged and therefore neutralize the negative charges of most shampoos and hair proteins. Their positive charges also ionically bond them to hair and skin.

Acrylates/C10-30 Alkyl Acrylate Crosspolymer is a copolymer of C10-30 alkyl acrylate and one or more monopolymers of acrylic acids, methacrylic acid, or one of their simple esters, cross-linked with an allyl ether of sucrose, or an allyl ether of pentaerythritol and is classed as a synthetic polymer. This cross-linked polyacrylic acid polymer is often used as an emulsion stabilizer as well as an aqueous or non-aqueous viscosity increasing agent. It is also a thickener useful for thickening surfactant systems and has excellent thickening efficiency and suspending capability, long viscous flow and sparkling clarity in gel systems. It is also used to make dispersions in water less susceptible to lumping and easier to pump and handle due to its low dispersion viscosity before neutralization.

It has been determined that an effective combination of the improved composition comprises from about 0.01% to 80% purified water, from about 0.01% to 60% sodium laureth sulfate, from about 0.01% to 20% zinc pyrithione, from about 0.01% to 20% cocamidopropyl betaine, from about 0.01% to 20% glycerol cocoate, from about 0.01% to 20% polyquaternium-7, and from about 0.01% to 20% of a cross-linked polyacrylic acid polymer. In another embodiment, the improved composition includes from about 30% to 70% purified water, from about 10% to 50% sodium laureth sulfate, from about 0.01% to 10% zinc pyrithione, from about 0.01% to 10% cocamidopropyl betaine, from about 0.01% to 10% glycerol cocoate, from about 0.01% to 10% polyquaternium-7, and from about 0.01% to 10% of a cross-linked polyacrylic acid polymer. In still another embodiment, the improved composition includes from about 40% to 60% purified water, from about 20% to 40% sodium laureth sulfate, from about 0.01% to 5% zinc pyrithione, from about 0.01% to 5% cocamidopropyl betaine, from about 0.01% to 5% glycerol cocoate, from about 0.01% to 5% polyquaternium-7, and from about 0.01% to 5% of a cross-linked polyacrylic acid polymer.

Additional active ingredients that may be included in the improved composition include from about 0.001% to 10% allantoin, from about 0.001 to 10% dimethicone copolymer, from about 0.001% to 10% sodium hydroxide, from about 0.001% to 10% phenoxyethanol, and from about 0.001% to 10% of one or more of the following preservatives: methylparaben, ethylparaben, propylparaben, butylparaben, and isobutyl paraben. In another embodiment, the improved composition includes from about 0.001% to 5% allantoin, from about 0.001 to 5% dimethicone copolymer, from about 0.001% to 5% sodium hydroxide, from about 0.001% to 5% phenoxyethanol, and from about 0.001% to 5% of one or more of the following preservatives: methylparaben, ethyleparaben, propylparaben, butylparaben, and isobutyl paraben. In still another embodiment, the improved composition includes from about 0.1% to 2% allantoin, from about 0.1% to 2% dimethicone copolymer, from about 0.1% to 2% sodium hydroxide, from about 0.1% to 2% phenoxyethanol, and from about 0.001% to 10% of one or more of the following preservatives: methylparaben, ethyleparaben, propylparaben, butylparaben, and isobutyl paraben.

Allantoin is a botanical extract of the comfrey plant and is often used for its healing, soothing, and anti-irritation properties. Allantoin is also known to aid in healing wounds, skin irritations and stimulate growth of healthy tissue. Allantoin is also known as 5-ureidohydantoin or glyoxyldiureide. It is a product of oxidation of uric acid and is a diureide of glyoxylic acid.

PEG-12 dimethicone, also known as dimethicone copolymer, is a very versatile, water-soluble, polyether modified silicone that is often used in skin and hair care products. Sodium hydroxide, also known as lye or caustic soda, is a caustic metallic base. An alkali, sodium hydroxide is widely used in many industries, mostly as a strong chemical base in the manufacture of pulp and paper, textiles, drinking water, and detergents. Sodium hydroxide is completely ionic, containing sodium ions and hydroxide ions. The hydroxide ion makes sodium hydroxide a strong base which reacts with acids to form water and the corresponding salts.

Phenoxyethanol is an organic chemical compound, a glycol ether often used in dermatological products such as skin creams. It is a colorless oily liquid. It also functions as a bactericide. Phenoxyethanol may also be used as a fixative for perfumes, an insect repellent, a topical antiseptic, a solvent for cellulose acetate, some dyes, inks and resins, in preservatives, pharmaceuticals, and in organic synthesis. It is moderately soluble in water.

Methylparaben, ethylparaben, propylparaben, butylparaben and isobutylparaben are all useful as preservative. Methylparaben is the methyl ester of p-hydroxybenzoic acid. It is a mold-inhibitor and often used as a preservative for food and cosmetics. Ethylparaben, also known as ethyl para-hydroxybenzoate, is the ethyl ester of p-hydroxybenzoic acid. Propylparaben, the propyl ester of p-hydroxybenzoic acid, occurs as a natural substance found in many plants and some insects although it is manufactured synthetically for use in cosmetics, pharmaceuticals and foods. Butylparaben is the butyl ester of p-hydroxybenzoic acid and isobutylparaben is the ester of isobutyl alcohol and p-hydroxybenzoic acid. It is classed as both an ester and phenol and is often used as a preservative.

The improved composition of the present invention may include one or more additional components without departing from the scope of the present invention. These components may include cosmeceuticals, anti-aging, rejuvenating or antioxidant therapies, antiinflammatory agents, as well as over-the-counter or prescription drugs, in various strengths as are known and pharmaceutically acceptable, depending on the desired ultimate formulation of the improved composition. It is further noted that the term “acid” includes pharmaceutically acceptable salts thereof.

The improved composition of the present invention may further include one or more non-active ingredients well known in the art, including surfactants, preservatives, excipients, gelling agents, fragrances, buffers, binders, emulsifiers, solvents, electrolytes, sebum absorbing polymers and other polymers, pigments, sunblocking and sunscreening agents, physical exfoliating particles, liposomes and chelators.

In an illustrative example, a suitable carrier is formulated, as is well known in the art, after which the active ingredients are added. Suitable carriers include emulsions, creams, lotions, ointments, serums, liquids, lacquers, gels, sprays, exfoliating particulates, cleansing agents, cosmetics agents, bath additives, oils, nanosized particulates or liposomes, fragrances, powders, muds and masks.

In an illustrative embodiment, the improved composition is applied topically to areas affected by a skin disorder and then rinsed off at least once, preferably at least once or twice daily, until improvement is observed, and then at least once or twice daily to maintain the improvement.

### EXAMPLE

TABLE 1
An Illustrative Example of the Improved Cleanser Composition

| Raw Materials | Wt/wt % |
|---|---|
| Purified Water | 50.5933 |
| Carbopol Ultrez 21 (cross-linked polyacrylic | 1.2000 |
| acid polymer) | |
| Allantoin (ISP) | 0.5000 |
| Sodium Hydroxide 100% NF/Fcc | 0.3487 |
| PEG-7 Glycerol Cocoate (UPI) | 3.0000 |
| Phenonip (Nipa) | 1.000 |
| Stepan STEOL CS-230 | 32.0000 |
| Amphosol CA - Cocamidopropyl Betaine | 4.000 |
| (Stepan) | |
| Merquat 550 (Nalco) | 3.000 |
| Botanisil S-19 (DDChemco) | 0.2000 |
| Zinc Omadine CG (Arch) | 4.1600 |

50.2466% purified water was heated to 45° C. 1.2000% Carbopol Ultrez 21 was sprinkled onto the heated water, allowed to hydrate, and then mixed until homogenous. 0.5000% allantoin was then added and mixed until in solution to form the first phase.

The first phase solution was then partially neutralized with a 50% sodium hydroxide solution composed of 0.0180% purified water and 0.0180% sodium hydroxide 100% to form the second phase. Next, 3.0000% PEG-7 Glyceryl Cocoate, 1.0000% Phenonip, and 32.0000% Stepan STEOL CS-230 was added to the second phase and mixed until homogenous to form the third phase.

The pH of the third phase was then adjusted to 5.8-6.0 with a 50% sodium hydroxide solution composed of 0.0180% purified water and 0.0180% sodium hydroxide 100% to form the fourth phase. Next, 4.000% Amphosol CA—Cocamidopropyl Betaine, 3.000% Merquat 550, and 0.200% Botanisil S-19 were added one-at-a-time to the fourth phase and mixed. 4.1600% Zinc Omadine CG was then added and mixed until completely dispersed. High speed agitation was used as necessary to ensure complete dispersion.

Clinical Trials

The resulting cleanser composition (test material) was tested for dryness, erythema, and mildness.

Panel Selection

Ten healthy male and female subjects (1 male and 9 females), ranging in age from 28 to 57 years, were impaneled for study participation. The subjects attested to being in good health, free of any significant active skin pathology involving the anatomical sites where the test material was applied. All subjects agreed to use no new topical agents, either physical or chemical, on the test area from one week prior to testing and for the duration of the clinical trial other than non-moisturizing cleansers and water. Subjects were allowed to bathe normally, avoiding the application areas. Exclusion criteria included: pregnancy or attempting same during study period, lactation, and presence of fissures, erosions, or inflammation of the skin at the application sites.

Test Method

The test material and a control product were packaged individually and labeled only as A or B to prevent identification by both test personnel and the subjects. At the commencement of testing, a randomization list was constructed using standard techniques. This list was used to guide the designation of which arm on each subject each product (A or B) was applied to. Subjects were placed on the randomization list in his or her order of appearance for the test.

The initial wash application was conducted by technicians according to the study protocol wherein one arm of a subject was washed with product A and the other arm of the subject was washed with product B. Subsequent washes were repeated on a daily basis at home by each subject. On the fourth day, the subjects were evaluated.

Results

Completed and Discontinued Subjects:

All ten subjects completed the study.

Study Data:

Graded symptoms were analyzed by means of a nonparametric Wilcoxon Signed Rank Test. On the fourth day, subjects were evaluated by the Principal Investigator of the clinical trial on a one to five scale for dryness and erythema on each arm. On that scale, a grade of one represented little to none of the named attribute and five represented intense appearance thereof. Additionally, the subjects evaluated each product for mildness. The differences in the grades of each symptomatic parameter were compared for differences between the test and control products which results are shown in Table I. No statistically significant differences were presented in the evaluated attributes of the skin of each arm. The results for the subjects' perception of mildness are presented in Table II. No statistically significant differences were presented in the evaluated attribute of mildness on the skin of each arm.

TABLE I
Nonparametric Significance - Observer Findings

| | Test vs. Control Arm | Test vs. Control Arm |
|---|---|---|
| | Differences - Dryness | Differences - Redness |
| Wilcoxon Z Value | −.577(a) | −.577(a) |
| Significance Level | .564 | .564 |
| (2-tailed) | | |

(a)Based on Negative Ranks
b. Wilcoxon Signed Ranks Test

TABLE II
Nonparametric Significance - Mildness

| | Test vs. Control Arm |
|---|---|
| | Differences - Mildness |
| Wilcoxon Z Value | −.333(a) |
| Significance Level (2-tailed) | .739 |

(a)Based on Negative Ranks
b. Wilcoxon Signed Ranks Test

No participants complained at any time during the study of symptoms attributable to adverse effects of either product, such as irritation, itching or stinging at the application sites. No participants were observed by study personnel to have skin abnormalities at application sites during the study.

Having described the invention in detail, those skilled in the art will appreciate that modifications may be made of the invention without departing from its spirit and scope. Therefore, it is not intended that the scope of the invention be limited to the specific embodiments described. Rather, it is intended that the appended claims and their equivalents determine the scope of the invention.

I claim:

1. A cleanser composition:
from about 0.01% to 80% purified water;
from about 0.01% to 60% sodium laureth sulfate;
from about 0.01% to 20% zinc pyrithione;
from about 0.01% to 20% cocamidopropyl betaine;
from about 0.01% to 20% glycerol cocoate;
from about 0.01% to 20% polyquaternium-7; and
from about 0.01% to 20% of a cross-linked polyacrylic acid polymer.

2. The cleanser composition of claim 1 further comprising:
from about 30% to 70% purified water;
from about 10% to 50% sodium laureth sulfate;
from about 0.01% to 10% zinc pyrithione;
from about 0.01% to 10% cocamidopropyl betaine;
from about 0.01% to 10% glycerol cocoate;
from about 0.01% to 10% polyquaternium-7; and
from about 0.01% to 10% of a cross-linked polyacrylic acid polymer.

3. The cleanser composition of claim 1 further comprising:
from about 40% to 60% purified water;
from about 20% to 40% sodium laureth sulfate;
from about 0.01% to 5% zinc pyrithione;
from about 0.01% to 5% cocamidopropyl betaine;
from about 0.01% to 5% glycerol cocoate;
from about 0.01% to 5% polyquaternium-7; and
from about 0.01% to 5% of a cross-linked polyacrylic acid polymer.

4. The cleanser composition of claim 1 further comprising:
from about 0.001% to 10% allantoin;
from about 0.001 to 10% dimethicone copolymer;
from about 0.001% to 10% sodium hydroxide;
from about 0.001% to 10% phenoxyethanol; and
from about 0.001% to 10% of a preservative selected from the group consisting of methylparaben, ethyleparaben, propylparaben, butylparaben, isobutyl paraben, and mixtures thereof.

5. The cleanser composition of claim 1 further comprising:
from about 0.001% to 5% allantoin;
from about 0.001 to 5% dimethicone copolymer;
from about 0.001% to 5% sodium hydroxide;
from about 0.001% to 5% phenoxyethanol; and
from about 0.001% to 5% of a preservative selected from the group consisting of methylparaben, ethyleparaben, propylparaben, butylparaben, isobutyl paraben, and mixtures thereof.

6. The cleanser composition of claim 1 further comprising:
from about 0.1% to 2% allantoin;
from about 0.1% to 2% dimethicone copolymer;
from about 0.1% to 2% sodium hydroxide;
from about 0.1% to 2% phenoxyethanol; and
from about 0.001% to 10% of a preservative selected from the group consisting of methylparaben, ethyleparaben, propylparaben, butylparaben, isobutyl paraben, and mixtures thereof.

7. The cleanser composition of claim 1 further comprising one or more ingredients selected from the group consisting of cosmeceuticals, anti-aging, rejuvenating or antioxidant therapies, antiinflammatory agents, over-the-counter pharmaceuticals, prescription pharmaceuticals, surfactants, preservatives, excipients, gelling agents, fragrances, buffers, binders, emulsifiers, solvents, electrolytes, sebum absorbing polymers and other polymers, pigments, sunblocking and sunscreening agents, physical exfoliating particles, liposomes and chelators.

8. The cleanser composition of claim 1 further comprising at least one pharmaceutical or salt thereof.

9. The cleanser composition of claim 1 further comprising a carrier selected from the group consisting of emulsions, creams, lotions, ointments, serums, liquids, lacquers, gels, sprays, exfoliating particulates, cleansing agents, cosmetics agents, bath additives, oils, nanosized particulates or liposomes, fragrances, powders, muds and masks.

10. A cleanser composition for treating a skin disorder:
from about 0.01% to 80% purified water;
from about 0.01% to 60% sodium laureth sulfate;
from about 0.01% to 20% zinc pyrithione;
from about 0.01% to 20% cocamidopropyl betaine;
from about 0.01% to 20% glycerol cocoate;
from about 0.01% to 20% polyquaternium-7; and
from about 0.01% to 20% of a cross-linked polyacrylic acid polymer.

11. The cleanser composition of claim 10 further comprising:
from about 30% to 70% purified water;
from about 10% to 50% sodium laureth sulfate;
from about 0.01% to 10% zinc pyrithione;
from about 0.01% to 10% cocamidopropyl betaine;
from about 0.01% to 10% glycerol cocoate;
from about 0.01% to 10% polyquaternium-7; and
from about 0.01% to 10% of a cross-linked polyacrylic acid polymer.

12. The cleanser composition of claim 10 further comprising:
from about 40% to 60% purified water;
from about 20% to 40% sodium laureth sulfate;
from about 0.01% to 5% zinc pyrithione;
from about 0.01% to 5% cocamidopropyl betaine;
from about 0.01% to 5% glycerol cocoate;
from about 0.01% to 5% polyquaternium-7; and
from about 0.01% to 5% of a cross-linked polyacrylic acid polymer.

13. The cleanser composition of claim 10 further comprising:
from about 0.001% to 10% allantoin;
from about 0.001 to 10% dimethicone copolymer;
from about 0.001% to 10% sodium hydroxide;
from about 0.001% to 10% phenoxyethanol; and
from about 0.001% to 10% of a preservative selected from the group consisting of methylparaben, ethyleparaben, propylparaben, butylparaben, isobutyl paraben, and mixtures thereof.

14. The cleanser composition of claim 10 further comprising:
from about 0.001% to 5% allantoin;
from about 0.001 to 5% dimethicone copolymer;
from about 0.001% to 5% sodium hydroxide;
from about 0.001% to 5% phenoxyethanol; and
from about 0.001% to 5% of a preservative selected from the group consisting of methylparaben, ethyleparaben, propylparaben, butylparaben, isobutyl paraben, and mixtures thereof.

15. The cleanser composition of claim 10 further comprising:
from about 0.1% to 2% allantoin;
from about 0.1% to 2% dimethicone copolymer;
from about 0.1% to 2% sodium hydroxide;
from about 0.1% to 2% phenoxyethanol; and
from about 0.001% to 10% of a preservative selected from the group consisting of methylparaben, ethyleparaben, propylparaben, butylparaben, isobutyl paraben, and mixtures thereof.

16. The cleanser composition of claim 10 further comprising one or more non-active ingredients selected from the group consisting of cosmeceuticals, anti-aging, rejuvenating or antioxidant therapies, antiinflammatory agents, over-the-counter pharmaceuticals, prescription pharmaceuticals, surfactants, preservatives, excipients, gelling agents, fragrances, buffers, binders, emulsifiers, solvents, electrolytes, sebum absorbing polymers and other polymers, pigments, sunblocking and sunscreening agents, physical exfoliating particles, liposomes and chelators.

17. The cleanser composition of claim 10 further comprising at least one pharmaceutical or salt thereof.

18. The cleanser composition of claim 10 further comprising a carrier selected from the group consisting of emulsions, creams, lotions, ointments, serums, liquids, lacquers, gels, sprays, exfoliating particulates, cleansing agents, cosmetics agents, bath additives, oils, nanosized particulates or liposomes, fragrances, powders, muds and masks.

19. The cleanser composition of claim 10 wherein said skin disorder is selected from the group consisting of seborrheic dermatitis, perioral dermatitis, rosacea, acne, tinea versicolor, eczema and psoriasis.

20. A method of making a cleanser composition for the treatment of a skin disorder, the method comprising the steps of:
forming a suitable carrier; and
adding from about 0.01% to 80% purified water;
from about 0.01% to 60% sodium laureth sulfate;
from about 0.01% to 20% zinc pyrithione;
from about 0.01% to 20% cocamidopropyl betaine;
from about 0.01% to 20% glycerol cocoate;
from about 0.01% to 20% polyquaternium-7; and
from about 0.01% to 20% of a cross-linked polyacrylic acid polymer to the suitable carrier to form a substantially homogeneous mixture.

21. The homogenous mixture of claim 20 further comprising:
from about 30% to 70% purified water;
from about 10% to 50% sodium laureth sulfate;
from about 0.01% to 10% zinc pyrithione;
from about 0.01% to 10% cocamidopropyl betaine;
from about 0.01% to 10% glycerol cocoate;
from about 0.01% to 10% polyquaternium-7; and
from about 0.01% to 10% of a cross-linked polyacrylic acid polymer.

22. The homogenous mixture of claim 20 further comprising:
from about 40% to 60% purified water;
from about 20% to 40% sodium laureth sulfate;
from about 0.01% to 5% zinc pyrithione;
from about 0.01% to 5% cocamidopropyl betaine;
from about 0.01% to 5% glycerol cocoate;
from about 0.01% to 5% polyquaternium-7; and
from about 0.01% to 5% of a cross-linked polyacrylic acid polymer.

23. The homogenous mixture of claim 20 further comprising:
from about 0.001% to 10% allantoin;
from about 0.001 to 10% dimethicone copolymer;
from about 0.001% to 10% sodium hydroxide;
from about 0.001% to 10% phenoxyethanol; and
from about 0.001% to 10% of a preservative selected from the group consisting of methylparaben, ethyleparaben, propylparaben, butylparaben, isobutyl paraben, and mixtures thereof.

24. The homogenous mixture of claim 20 further comprising:
from about 0.001% to 5% allantoin;
from about 0.001 to 5% dimethicone copolymer;
from about 0.001% to 5% sodium hydroxide;
from about 0.001% to 5% phenoxyethanol; and
from about 0.001% to 5% of a preservative selected from the group consisting of methylparaben, ethyleparaben, propylparaben, butylparaben, isobutyl paraben, and mixtures thereof.

25. The homogenous mixture of claim 20 further comprising:
from about 0.1% to 2% allantoin;
from about 0.1% to 2% dimethicone copolymer;
from about 0.1% to 2% sodium hydroxide;
from about 0.1% to 2% phenoxyethanol; and
from about 0.001% to 10% of a preservative selected from the group consisting of methylparaben, ethyleparaben, propylparaben, butylparaben, isobutyl paraben, and mixtures thereof.

26. The method of claim 20 further comprising the step of adding one or more non-active ingredients selected from the group consisting of cosmeceuticals, anti-aging, rejuvenating or antioxidant therapies, antiinflammatory agents, over-the-counter pharmaceuticals, prescription pharmaceuticals, surfactants, preservatives, excipients, gelling agents, fragrances, buffers, binders, emulsifiers, solvents, electrolytes, sebum absorbing polymers and other polymers, pigments, sunblocking and sunscreening agents, physical exfoliating particles, liposomes and chelators.

27. The method of claim 20 further comprising the step of adding at least one pharmaceutical or salt thereof.

28. The carrier of claim 20 being selected from the group consisting of emulsions, creams, lotions, ointments, serums, liquids, lacquers, gels, sprays, exfoliating particulates, cleansing agents, cosmetics agents, bath additives, oils, nanosized particulates or liposomes, fragrances, powders, muds and masks.

29. The method of claim 20 wherein said skin disorder is selected from the group consisting of seborrheic dermatitis, perioral dermatitis, rosacea, acne, tinea versicolor, eczema and psoriasis.

30. A method of treating a skin disorder, the method comprising topically administering to an affected area a non-toxic effective dosage of a composition comprising:
from about 0.01% to 80% purified water;
from about 0.01% to 60% sodium laureth sulfate;
from about 0.01% to 20% zinc pyrithione;
from about 0.01% to 20% cocamidopropyl betaine;
from about 0.01% to 20% glycerol cocoate;
from about 0.01% to 20% polyquaternium-7; and
from about 0.01% to 20% of a cross-linked polyacrylic acid polymer.
